(19) United States
(12) Patent Application Publication
(10) Pub. No.: US 2019/0360933 A1
(43) Pub. Date: Nov. 28, 2019

(54) METAL-ORGANIC FRAMEWORKS AS PROTECTIVE COATINGS AND FOR ENHANCING SENSITIVITY OF BIODIAGNOSTIC CHIPS

(71) Applicant: WASHINGTON UNIVERSITY IN ST. LOUIS, St. Louis, MO (US)

(72) Inventors: Srikanth Singamaneni, St. Louis, MO (US); Jeremiah J. Morrissey, St. Louis, MO (US); Evan D. Kharasch, St. Louis, MO (US); Qisheng Jiang, St. Louis, MO (US); Congzhou Wang, St. Louis, MO (US); Jingyi Luan, St. Louis, MO (US)

(21) Appl. No.: 16/461,725

(22) PCT Filed: Nov. 17, 2017
(86) PCT No.: PCT/US2017/062283

§ 371 Date: May 16, 2019

Related U.S. Application Data
(60) Provisional application No. 62/423,812, filed on Nov. 18, 2016.

(51) Int. Cl. G01N 21/552; G01N 21/65; G01N 33/543

(52) U.S. Cl. CPC G01N 21/554; G01N 33/54373; G01N 21/658

(57) ABSTRACT

Disclosed are diagnostic reagents having metal-organic frameworks and protection and enhancement of diagnostic reagents and materials using metal-organic frameworks.

### CROSS REFERENCE TO RELATED APPLICATIONS

This application claims priority to U.S. Provisional Patent Application Ser. No. 62/423,812, filed on Nov. 18, 2016, the disclosure of which is hereby incorporated by reference in its entirety.

### STATEMENT REGARDING FEDERALLY SPONSORED RESEARCH OR DEVELOPMENT

This invention was made with government support under DK100759 and CA141521 awarded by the National Institutes of Health, CBET1254399 awarded by the National Science Foundation, and FA9550-15-1-0228 awarded by Air Force Office of Scientific Research. The Government has certain rights in the invention.

### BACKGROUND OF THE DISCLOSURE

The present disclosure relates generally to protection and enhancing sensitivity of diagnostic reagents and materials. More particularly, the present disclosure relates to the protection and enhancement of diagnostic reagents and materials using metal-organic frameworks.

Metal-organic frameworks (MOFs), composed of metal ions or clusters linked by organic ligands, have received increased scientific and technological interest due to their large surface area, tunable porosity and organic functionality, as well as high thermal stability. These attractive properties render MOFs promising materials for a variety of applications in gas storage, drug delivery, catalysis, and chemical sensors. Within the different emerging applications, a recent study demonstrated the encapsulation of a wide range of biomolecules within MOFs by growing them in the presence of the biomolecules under mild biocompatible conditions (for instance, in aqueous solution at room temperature). More importantly, with the protection of MOF layer, the activity of encapsulated biomolecules (such as enzymes) could be preserved against different extreme environmental conditions including high temperatures and organic solvents. This work demonstrated the preservation of biocatalytic activity of enzymes in solution.

Antibody-antigen interactions form the basis for various conventional bioassays including enzyme-linked immunosorbent assay (ELISA), immunoblotting and immunoprecipitation assays, owing to their superior binding affinity and selectivity. Recent years, with the rapid development and wide application of biomedical diagnostic tools such as lab-on-a-chip biosensors, antibodies have also been ubiquitously employed as target recognition elements in the biosensors with different types of transduction platforms (electrochemical, magnetic, or optical). Unfortunately, as protein molecules, the major limitation of antibodies lies in their poor stability at elevated temperatures and in non-aqueous media (for instance, on transducer surfaces after immobilization). Thus, the antibody-based diagnostic reagents and biosensor chips are required to be maintained under tightly regulated refrigerated conditions, to preserve their bio-functionality (recognition capability). This stringent requirement necessitates a temperature-controlled supply chain, the “cold chain”, during transport, storage, and handling of the biodiagnostic reagents and biosensor chips. Apart from causing huge financial and environmental burden, the cold chain system is simply not feasible in pre-hospital and resource-limited settings such as developing countries, disaster struck regions, and battle field, where refrigeration and electricity are not guaranteed. Accordingly, there exists a need for alternate approaches to preserve the biorecognition capability that relaxes or eliminates the cold chain requirement.

Artificial antibodies based on molecular imprinting presents an attractive alternative to natural antibodies due to their low cost and high stability. The sensitivity of artificial antibodies, however, is inferior compared to natural antibodies. Accordingly, there exists a need to increase the sensitivity of platforms based on artificial antibody-based sensors.

### BRIEF DESCRIPTION

In one aspect, the present disclosure is directed to a metal-organic framework protected plasmonic sensor comprising a nanostructure core, a biomolecule, and a metal-organic framework.

In one aspect, the present disclosure is directed to a plasmonic device comprising a substrate, a plasmonic sensor and a metal-organic framework.

In one aspect, the present disclosure is directed to a metal-organic framework enhanced plasmonic sensor comprising a molecular imprinted polymer plasmonic sensor, the molecular imprinted polymer plasmonic sensor comprising a nanostructure core and functional monomers polymerized to the nanostructure core, wherein the polymerized functional monomers comprise at least one recognition cavity that is substantially complementary to a target analyte; a target analyte, and a metal-organic framework.

In another aspect, the present disclosure is directed to a method for preparing a metal-organic framework protected plasmonic sensor comprising a nanostructure core, a biomolecule conjugated to the nanostructure core, and a metal-organic framework. The method comprises: conjugating the biomolecule to the nanostructure core to form a nanostructure core-biomolecule complex; and contacting the nanostructure core-biomolecule complex with a metal-oxide framework precursor solution for a time sufficient to form a metal-organic framework.

In another aspect, the present disclosure is directed to a method for preparing a thermally stable plasmonic device. The method comprises: conjugating a biomolecule to a nanostructure core to form a nanostructure core-biomolecule complex; adhering the nanostructure core-biomolecule complex to a substrate; and contacting the substrate with a metal-oxide framework precursor solution for a time sufficient to form a metal-organic framework.

In another aspect, the present disclosure is directed to a method for enhancing thermal stability and preserving recognition capability of a biomolecule on biosensor device surface. The method comprises: conjugating the biomolecule to a nanostructure core to form a nanostructure core-biomolecule complex; adhering the nanostructure core-biomolecule complex to a substrate; and contacting the substrate with a metal-oxide framework precursor solution for a time sufficient to form a metal-organic framework.

In another aspect, the present disclosure is directed to a method for enhancing a signal produced in a label-free detection method. The method comprises: contacting a sample with a plasmonic sensor, wherein the plasmonic sensor comprises a nanostructure core and functional monomers polymerized to the nanostructure core, wherein the polymerized functional monomers comprise at least one recognition cavity that is substantially complementary to a target analyte; wherein a target analyte in the sample forms a complex with the plasmonic sensor; incubating the complex in a metal-organic framework precursor solution and; detecting the complex.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

FIG. 1 is an illustration depicting the concept of using MOF to enhance the thermal stability of antibody-based plasmonic biochips, not only eliminating the need for refrigerated transportation, handling and storage, but also enabling convenient use in resource limited settings.

FIG. 2A is a TEM image of AuNRs used as plasmonic nanotransducers. The dimension of the AuNRs is 48×18 nm.

FIG. 2B is a graph depicting the extinction spectra showing the LSPR shift of AuNR (dashed line) after conjugation of AuNR with IgG in solution (solid line). The λₘₐₓ red shifts by 7.5 nm.

FIG. 2C is a graph depicting the LSPR shift of AuNR-IgG on a glass substrate upon exposure to various concentrations of anti-IgG solutions showing the monotonic increase in LSPR shift with concentration. Error bars represent standard deviations from three different samples.

FIG. 2D is a graph depicting the extinction spectra of AuNR-IgG conjugates on a glass substrate before (dashed line) and after exposure to anti-IgG (24 μg/ml) (solid line). The λₘₐₓ red shifts by 15.5 nm.

FIG. 3A is a graph depicting the extinction spectra of AuNR-IgG conjugates on the glass substrate before and after MOF film coating, after rinsing MOF film and after exposure to 24 μg/ml of anti-IgG.

FIG. 3B is a graph depicting the LSPR shift corresponding to each step in FIG. 3A.

FIG. 3C is an atomic force microscopy (AFM) image showing uniformly adsorbed AuNR-IgG on glass substrate before MOF film coating.

FIG. 3D is an AFM image showing AuNR-IgG conjugates covered by MOF film.

FIG. 3E is an AFM image showing AuNR-IgG conjugates following MOF film removal.

FIG. 3F is a graph depicting FTIR spectra of AuNR-IgG before and after MOF coating demonstrating the complete removal of MOF after rinsing with water at pH 6.

FIG. 3G is a graph depicting X-ray diffraction spectra of AuNR-IgG before and after MOF coating.

FIG. 4A is a graph depicting the retained recognition capability of MOF-coated IgG-AuNR conjugates on glass substrates stored at room temperature, 40° C. and 60° C. for different durations.

FIG. 4B is a graph depicting the comparison of preservation efficiency between MOF and silk as the protective materials after one week at room temperature and 40° C. Error bars represent standard deviations from three samples.

FIG. 5A are SEM images showing AuNR-anti-NGAL conjugates uniformly adsorbed on a paper substrate. Inset at the top left shows images of the bare filter paper (left) and filter paper after adsorption of AuNR-NGAL antibody conjugates (right). Inset at the top right shows the higher magnification image of paper with AuNR-anti-NGAL conjugates.

FIG. 5B is a graph depicting the extinction spectra of AuNR-anti-NGAL conjugates on the paper substrate before and after MOF film coating, after MOF film rinsing and after exposure to 2.5 μg/ml of NGAL.

FIG. 5C is a graph depicting the LSPR shift corresponding to each step in FIG. 5B.

FIG. 5D is a graph depicting the Retained recognition capability of MOF-coated IgG-anti-NGAL conjugates on paper substrates stored at room temperature, 40° C. and 60° C. for different durations. Error bars represent standard deviations from three samples.

FIG. 6 is a schematic illustrating the various steps involved in fabrication of the MOF amplified MIP-based plasmonic biosensor, target protein capture, followed by the mineralization of MOF around the captured protein to enhance the LSPR signal.

FIG. 7A depicts an AFM height images of HSA absorbed on HOPG substrate (height scale: 15 nm).

FIG. 7B depicts an AFM height images of HSA after ZIF-8 growth (height scale: 30 nm) (Corresponding height histograms shown in FIG. 7C and FIG. 7D).

FIG. 7C is a graph depicting height histogram of HSA absorbed on HOPG substrate shown in FIG. 7A.

FIG. 7D is a graph depicting height histogram of HSA after ZIF-8 growth shown in FIG. 7B.

FIG. 7E depicts an AFM height images showing AuNR absorbed with HSA.

FIG. 7F depicts an AFM height images showing AuNR absorbed with HSA after ZIF-8 growth showing surface with significantly higher roughness.

FIG. 7G is a graph depicting the LSPR shift induced by the formation of MOF vs. growth time on HSA modified AuNR and bare AuNR.

FIG. 8A is a graph depicting the Raman spectra of HSA before (HSA) exposure to ZIF-8 precursor and after (HSA-MOF) exposure to ZIF-8 precursor.

FIG. 8B is a graph depicting x-ray diffraction of HSA before (HSA) exposure to ZIF-8 precursor and after (HSA-MOF) exposure to ZIF-8 precursor.

FIG. 9A is a TEM image of AuNR used as nanotransducers for artificial antibody-based biosensor.

FIG. 9B is a graph depicting Vis-NIR extinction spectra of aqueous suspension of AuNR (inset shows the size histogram of AuNR obtained from TEM image, revealing the average length of AuNR˜49.5 nm).

FIG. 9C depicts the size histogram of AuNR obtained from TEM image, revealing the average length of AuNR˜49.5 nm.

FIG. 9D is a graph depicting the extinction spectra exhibiting a continuous red shift of LSPR wavelength of MIP-AuNR biosensor after protein capture, and ZIF-8 mineralization.

FIG. 9E is an enlargement of the curve in FIG. 9D between 620 nm and 700 nm.

FIG. 9F depicts the LSPR shift of AuNR after each step along the fabrication of MIP, target protein capture, and MOF enhancement.

FIG. 10A is a graph depicting the LSPR shift vs. concentration of the target protein biomarker (HSA) before and after MOF amplification.

FIG. 10B is a graph depicting a concentration dependent LSPR shift for normal AuNR-MIP and for MOF amplified AuNR-MIP at low target protein concentration.

FIG. 10C is a graph depicting the LSPR shift induced by the formation of MOF vs. MOF growth on molecularly imprinted AuNR before and after target protein capture.

FIG. 10D is a graph depicting the MOF amplification of MIP-based plasmonic for three different proteins biosensors showing the generality of the signal amplification strategy.

FIG. 11A is a graph depicting the timeline of the IgM and IgG as a model Flavivirus infection.

FIG. 11B is a schematic illustrating the progressive shift in the LSPR peak wavelength of gold nanotransducers (step a) following functionalization (step b) and following analyte binding (step c).

FIG. 11C is a graph depicting antibody binding measured in steps (a)-(c) in FIG. 11B by a red-shift of the LSPR of the AuNR.

FIG. 11D depicts the homogeneity of the nanorod preparation prior to functionalization with recombinant Zika NS1 protein “bait” in terms of image by TEM and measurements of widths and lengths of TEM images.

FIG. 11E is a graph depicting the shift in the LSPR peak of Zika NS1 protein-functionalized gold nanorods (ZikaNS1-AuNR) with increasing concentration of a monoclonal IgG specific for Zika NS1 protein. Mean±SD, n=10.

FIG. 11F is a graph depicting the concentration of IgM/IgG in control Zika-naive individuals and two Zika-infected patients.

FIG. 11G is a graph depicting the protection of Zika NS1 functionalized AuNR by MOF.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

In accordance with the present disclosure, plasmonic biosensors having a metal-organic framework and methods for preparing the plasmonic biosensors having a metal-organic framework are described. The plasmonic biosensors having a metal-organic framework provide a protective material to preserve the antibody recognition capability of biochips under elevated temperatures. Also described are plasmonic biosensors having a metal-organic framework and methods for preparing the plasmonic biosensors having a metal-organic framework. plasmonic biosensors having a metal-organic framework provide signal enhancement of LSPR signals.

### MOF Protected Plasmonic Sensor

In one aspect, the present disclosure is directed to a metal-organic framework protected plasmonic sensor. The plasmonic sensor includes a nanostructure core, a biomolecule, and a metal-organic framework.

Suitable nanostructure cores include nanospheres, nanorods, nanotubes, nanocubes, a nanobipyramids, nanocages, nanostars, nano-octahedrons, nanorattles, nanoshells, nanomatryoshkas, and combinations thereof. Suitable nanostructure cores also include hollow nanostructure cores including hollow nanocages, hollow hollow nanorattles, hollow nanoshells, and hollow nanomatryoshkas. Suitable nanostructure cores include metal nanoparticles. Particularly suitable metal nanostructure cores include gold nanostructure cores, silver nanostructure cores, copper nanostructure cores, and combinations thereof. Hollow nanostructure cores include metal nanostructure cores further including a porous metal shell. Hollow nanostructure cores can be prepared by coating a nanostructure core (e.g., nanoparticles, nanocubes, nanorods, nanobipyramids, nanostars, and nano-octahedra) with a metal to form a metal shell surrounding the nanostructure core. The metal shell can then be treated to form pores in the metal shell, and result in the formation of the hollow nanotransducer. In an exemplary aspect, a gold nanostructure core such as nano-octahedra can be coated with silver to form a bi-metallic core-shell nanostructure having a silver metal shell on the gold nanostructure core. The silver metal shell can then be treated such as using galvanic replacement reaction to convert the silver metal shell into a porous gold shell. The nanostructure core remains embedded in the porous shell. Average pore size in the shell can be about 3 nm. Pore sizes can be determined using transmission electron microscopy.

Biomolecules include nucleic acids such as DNA and RNA, full-length proteins, polypeptides, peptides, lipids, glycolipids, aptamers, and combinations thereof. Particularly suitable proteins include antibodies and antibody fragments. As well-known to those skilled in the art an antibody is a protein that recognizes and binds a specific antigen and that is generated by an immune system. Antibodies include monoclonal antibodies (i.e., consisting of identical antibody molecules), polyclonal antibodies (i.e., consisting of two or more different antibodies reacting with the same or different epitopes on the same antigen or even on distinct, different antigens), dimers, polymers, multi-specific antibodies (for example: double-specific antibody), and antibody fragments displaying target biological activities. Antibody fragments also includes chimeric and single chain antibodies, as well as binding fragments of antibodies, such as Fab, Fv fragments or single chain Fv (scFv) fragments.

A particularly suitable method for conjugating the biomolecule to the nanostructure core is by conjugating the biomolecule with a bifunctional polyethylene glycol (COOH-PEG-SH) chain and subsequently attaching the formed biomolecule-PEG-SH onto the nanostructure core surface through a gold-sulfur (Au—S) linkage. The PEG chain can serve as a flexible spacer, increase the accessibility of biomolecule to target analytes and can minimize non-specific binding. Conjugating the biomolecule onto the nanostructure core can be monitored by red shift of the longitudinal LSPR wavelength of the nanostructure core due to the increase in the refractive index of the medium surrounding the nanostructure core. MOF film formation can also be monitored by atomic force microscope (AFM) imaging.

Metal-organic frameworks (MOFs) include metal ions or clusters linked by organic ligands. Suitable metal-organic frameworks include zeolitic imidazolate framework-8 (ZIF-8) metal organic frameworks.

Plasmonic Device with MOF Protected Plasmonic Sensor

In another aspect, the present disclosure is directed to a plasmonic device comprising a substrate, a metal-organic framework, and a plasmonic sensor.

Suitable substrates include paper substrates, glass substrates, fibrous mats, plastic substrates, or any other surface capable of having nanostructures adsorb to the surface.

Suitable paper substrates include cellulose paper, nitrocellulose paper, methylcellulose paper, hydroxypropylcellulose paper, and nanocellulose paper. Suitable fibrous mats can be, for example, a woven fibrous mat and a non-woven fibrous mats. In one embodiment, the paper substrate can be immersed in a solution including the nanostructure core-biomolecule conjugates. The paper substrate having the nanostructure core-biomolecule conjugates can then be immersed in a MOF precursor solution. In another embodiment, the paper substrate can be immersed in a solution including plasmonic sensors having a nanostructure core, a biomolecule, and a metal-organic framework.

Suitable glass substrates include 3-mercaptopropyl)trimethoxysilane-modified glass substrates, poly(2-vinylpyridine) modified glass substrates and poly(styrene sulfonate) modified glass substrates. After adsorbing the nanostructure core-biomolecule conjugates to the glass substrate, the glass substrate having the nanostructure core-biomolecule conjugates can then be immersed in a MOF precursor solution.

In one embodiment, the plasmonic sensor having a biomolecule is adhered to the substrate and the metal-organic framework is a film coating. The metal-organic framework film coating can be formed by immersing nanostructure core-biomolecule conjugates into a metal-organic framework precursor solution. A suitable metal-organic framework precursor solution includes mixture of 2-methylimidazole and zinc acetate dihydrate. Metal-organic framework film formation can be monitored by detecting the LSPR wavelength shift, by atomic force microscope (AFM) imaging, Fourier transform infrared spectroscopy (FTIR), X-ray diffraction (XRD) and combinations thereof.

In another embodiment, the plasmonic sensor having a biomolecule further includes a metal-organic framework, which is then adhered to the substrate.

The biomolecule can be conjugated with the nanostructure core as described herein. Conjugating the biomolecule onto the nanostructure core can be monitored by red shift of the longitudinal LSPR wavelength of the nanostructure core due to the increase in the refractive index of the medium surrounding the nanostructure core. The nanostructure core-biomolecule conjugates can then be adsorbed to the substrate as described herein.

The plasmonic sensor includes a nanostructure core, as described herein. The plasmonic sensor includes a biomolecule, as described herein. The plasmonic sensor includes a metal-organic framework, as described herein.

Suitable metal-organic frameworks include zeolitic imidazolate framework-8 (ZIF-8) metal organic frameworks, as described herein.

The plasmonic device having the MOF protective layer can be stored until use. Advantageously, the plasmonic device of the present disclosure does not require storage being kept within a “cold chain” system. The plasmonic device of the present disclosure can be stored at stored at ambient and elevated temperatures.

The MOF protective coating can be removed by rinsing the plasmonic device with water. The removal of the MOF can be monitored by detecting the LSPR wavelength shift, by atomic force microscope (AFM) imaging, Fourier transform infrared spectroscopy (FTIR), X-ray diffraction (XRD) and combinations thereof. Removing the MOF protective coating before exposure to the analyte solution exposes the biomolecule to restore the recognition capability for the analyte.

Preparing Plasmonic Sensors with Artificial Antibodies

In one aspect, the present disclosure is directed to a metal-organic framework enhanced plasmonic sensor comprising a molecular imprinted polymer plasmonic sensor, the molecular imprinted polymer plasmonic sensor comprising a nanostructure core and functional monomers polymerized to the nanostructure core, wherein the polymerized functional monomers comprise at least one recognition cavity that is substantially complementary to a target analyte; a target analyte, and a metal-organic framework.

Plasmonic sensors with nanostructure cores and molecular imprinted polymer (also referred to interchangeably herein as “artificial antibodies”) can be achieved using a molecular imprinting approach. Nanostructures can be surface-imprinted using functional monomers to prepare plasmonic sensors with artificial antibodies (i.e., recognition cavities) as described herein. Polymerization of functional monomers such as silane and acrylamide on nanostructure cores having immobilized template molecules create a molecular imprinted polymer (MIP) film surrounding the nanostructure core and the immobilized template molecules. The fine control of the MIP film thickness as analyzed by LSPR spectroscopy and the non-uniform distribution of the capping ligand around the nanostructure cores can be exploited to favor molecular imprinting in areas that can be potent for plasmonic biosensing. In an aspect, the organo-siloxane monomers, siloxane monomers trimethoxypropylsilane (TMPS) and (3-aminopropyl)trimethoxysilane (APTMS) can be co-polymerized on the nanostructure surface with silane and acrylamide. Use of APTMS and TMPS in aqueous media provides a polymer with amine (NH₃⁺), hydroxyl (OH) and methyl (CH₃) functional groups. Without being limited to a particular theory, concerted weak interactions such as, electrostatic, hydrogen bonding and hydrophobic interactions, are believed to be the form of interaction in recognition cavity-target molecule complexes. The composition ratio of the co-polymerization can be adjusted to result in flexibility and mechanical strength of the polymer.

Suitable nanostructure cores are those described herein.

Suitable functional monomers are selected from a silane, acrylamide and combinations thereof. Suitable silanes can be selected from an organic silane monomer, 3-aminopropyltrimethoxysilane, propyltrimethoxysilane, benzyltriethoxysilane, benzyldimethylchlorosilane, acetamidopropyltrimethoxysilane, and combinations thereof.

In an aspect, the method can further include adsorbing to the functional monomers a molecule selected from polyethylene glycol and albumin prior to removing the template molecule.

Silane polymerization can be performed by sol-gel methods, where the polysiloxane networks are formed by siloxane bonds between silanol groups. The surface of the silica shells of silica-coated nanostructures can be modified by introducing amine functionality using 3-aminopropyltrimethoxysilane (APTMS). Aldehyde functionality can then be introduced using glutaraldehyde where the amine groups on the silica surface are converted to aldehyde groups. The template molecule can be bound to the surface of the silica-coated nanostructures using the aldehyde groups on the surface of these nanostructures, forming imine bonds. Following immobilization of the template molecule on the surface of the nanostructures, organic silane monomers, APTMS and propyltrimethoxysilane (PTMS) can be polymerized onto the surface at room temperature under buffered conditions to prevent the denaturing of the template molecules.

The template molecule is removed from the template molecule-nanostructure core structure by exposing the template molecule-nanostructure core structure to a reagent selected from an organic acid, an acid reagent, a detergent, and combinations thereof. Suitable reagents can be oxalic acid, sodium dodecyl sulfate (SDS), and combinations thereof. Oxalic acid breaks the imine bond between the silica surface and the template molecule, thus removing the template molecule from the surface. Removal of the template results in recognition cavities within the polymer, which act as specific recognition sites for the rebinding of the template molecule and a target molecule. The possibility of non-specific binding of the molecularly imprinted nanostructures is contemplated. To overcome this issue, polyethylene glycol (PEG) and albumin be used as a passivating layer. In one aspect, the polymer network can be formed using silane monomers functionalized with PEG side chains or albumin. In another aspect, PEG chains or albumin can be grafted to the polymer network before the template molecule is removed.

The template molecule can be a cell, a protein, a peptide, a nucleic acid, and combinations thereof. Similarly, the target analyte can be a cell, a protein, a peptide, a nucleic acid, and combinations thereof.

After the plasmonic sensors are formed, the plasmonic sensors can capture a target analyte. Following capture of the target analyte, the plasmonic sensor-target analyte complex is incubated in a metal-organic framework precursor solution. Incubation in a metal-organic framework precursor solution results in the formation of a metal-organic framework around the target analyte, and thus, forming the metal-organic framework enhanced plasmonic sensor comprising a molecular imprinted polymer plasmonic sensor, the molecular imprinted polymer plasmonic sensor comprising a nanostructure core and functional monomers polymerized to the nanostructure core, wherein the polymerized functional monomers comprise at least one recognition cavity that is substantially complementary to a target analyte; a target analyte, and a metal-organic framework.

### Methods for Enhancing a Signal Produced in a Label-Free Detection Method

In another aspect, the present disclosure is directed to a method for enhancing a signal produced in a label-free detection method. The method includes contacting a sample with a plasmonic sensor, wherein the plasmonic sensor comprises a nanostructure core and functional monomers polymerized to the nanostructure core, wherein the polymerized functional monomers comprise at least one recognition cavity that is substantially complementary to a target analyte; herein a target analyte in the sample forms a complex with the plasmonic sensor; incubating the complex in a metal-organic framework precursor solution and; detecting the complex.

The method of claim 13, wherein the complex is detected using a method selected from the group consisting of local surface plasmon resonance and surface enhanced Raman scattering.

The target analyte can be a cell, a protein, a peptide, a nucleic acid, and combinations thereof.

The plasmonic sensor can be adsorbed onto a surface. The surface can be, for example, glass, paper, plastic, or any other surface capable of having nanostructures adsorb to the surface. In an aspect, the surface can be a glass surface.

The sample can be a liquid biological sample. In an aspect, the liquid biological sample is selected from the group consisting of whole blood, plasma, serum, urine, saliva, cerebrospinal fluid, and sweat. In an aspect, the liquid biological sample can be a cell extract such as a cell homogenate.

The disclosure will be more fully understood upon consideration of the following non-limiting Examples.

### EXAMPLES

Gold nanorods were synthesized using a seed-mediated approach. Seed solution was prepared by adding 0.6 mL of an ice-cold solution of 10 mM sodium borohydride into 10 mL of vigorously stirred 0.1 M cetyltrimethylammonium bromide (CTAB) and 2.5×10⁻⁴ M HAuC₁₄ aqueous solution at room temperature. The color of the seed solution changed from yellow to brown. Growth solution was prepared by mixing 95 mL of 0.1 M CTAB, 1.0 mL of 10 mM silver nitrate, 5 mL of 10 mM HAuC₁₄, and 0.55 mL of 0.1 M ascorbic acid in the same order. The solution was homogenized by gentle stirring. To the resulting colorless solution, 0.12 mL of freshly prepared seed solution was added and set aside in dark for 14 hours. Prior to use, the AuNRs solution was centrifuged at 13,000 rpm for 10 minutes to remove excess CTAB and redispersed in nanopure water.

### Conjugation of Antibody and Protein to AuNR

Conjugation of the antibody (IgG) to AuNRs was achieved by conjugating IgG molecules with a bifunctional polyethylene glycol (COOH-PEG-SH) chain and subsequently attaching the formed IgG-PEG-SH onto the AuNR surface through an Au—S linkage. The PEG chain, serves as a flexible spacer, increases the accessibility of IgG to target biomolecules and also forms a stable protective layer around AuNRs to minimize non-specific binding.

Conjugation of Zika NS-1 protein was achieved with a bifunctional polyethylene glycol (COOH-PEG-SH) chain and subsequently attaching the formed Zika NS1-PEG-SH onto the AuNR surface through an Au—S linkage. The PEG chain, serves as a flexible spacer, increases the accessibility of Zika NS1 to target biomolecules and also forms a stable protective layer around AuNRs to minimize non-specific binding.

### Preparation of Molecularly Imprinted AuNR

To prepare molecularly imprinted AuNRs protein templates were immobilized on AuNR by exposing the AuNRs to a mixture of p-aminothiophenol (p-ATP) and glutaraldehyde (GA) (p-ATP/GA). In aqueous solutions, p-ATP binds spontaneously to gold surfaces with its thiol group, while GA molecules form oligomers of variable size with a free aldehyde group at each end of the oligomer molecules. As a result, GA functions as a cross linker between the amine groups of p-ATP molecules and the amine moieties on the side chains of the protein template molecules by forming unstable imine bonds in basic pH buffer solution.

Following the immobilization of the template, the organo-siloxane monomers trimethoxypropylsilane (TMPS) and (3-aminopropyl)trimethoxysilane (APTMS) were co-polymerized onto the modified AuNR surface. The subsequent condensation of the transient silanol groups yields an aminopropyl-functional amorphous polymer and entrapment of the protein templates. After rinsing, the protein-coated substrates were immersed in 3 mL phosphate buffer (pH 7.5) to which 5 μL TMPS and 5 μL APTMS were freshly added. The samples were then gently rinsed with PBS solution (pH 7.5) and stored in the same buffer overnight at 4° C.

The last step of the molecular imprinting process was the template release by breaking the imine bonds of the cross linker using a mixture of sodium dodecyl sulfate and oxalic acid. The protein template molecules inside the siloxane co-polymer were extracted by exposing the imprinted substrates to a mixture of 2% SDS and 2 mM oxalic acid for 1 h.

### Example 1

In this Example, MOF enhancement of the thermal stability of antibody-based plasmonic biochips was analyzed against ambient and elevated temperatures.

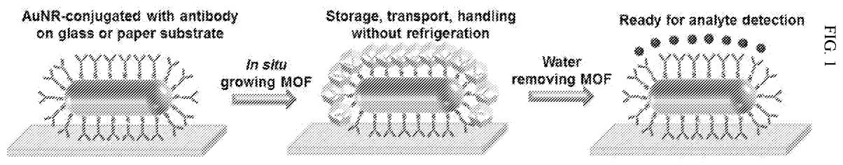

As illustrated in FIG. 1, gold nanorod (AuNR) conjugated with antibodies were adsorbed onto a glass substrate and a paper substrate. Just prior to using the biochip, a simple aqueous rinsing step completely removed the MOF protective layer and restored the biofunctionality of the sensor surface.

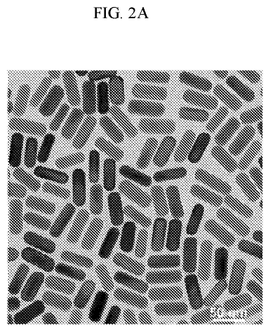

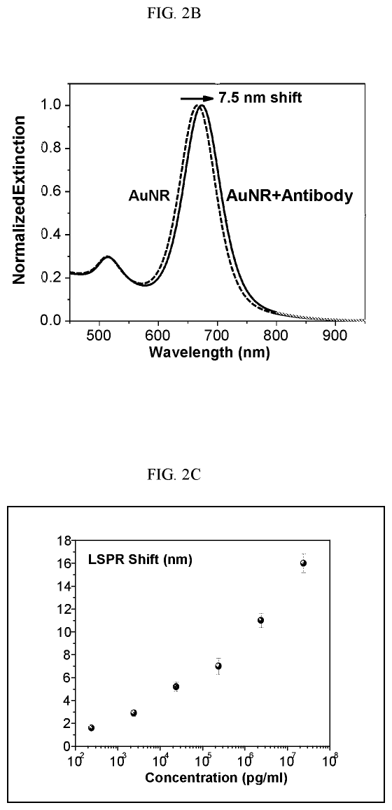

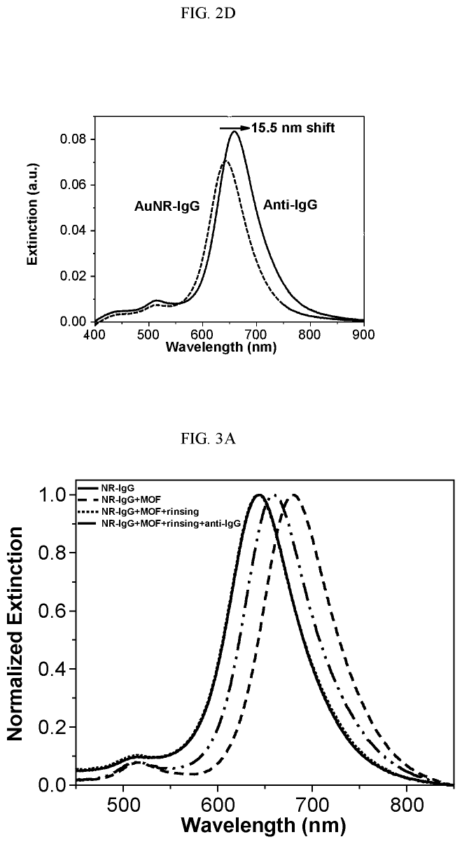

Rabbit IgG and goat anti-Rabbit IgG (termed IgG and anti-IgG henceforth) were employed as model antibody and bioanalyte, respectively, to establish the proof-of-concept. Here, gold nanorods (AuNRs) were used as plasmonic nanotransducers for label-free sensing because of their large refractive index sensitivity and excellent tunability of the LSPR wavelength. AuNRs were synthesized using a seed-mediated approach with a length of 48.2±1.8 nm and a diameter of 18.2±1.1 nm (FIG. 2A). The conjugation of the antibody (IgG) to AuNRs was achieved by conjugating IgG molecules with a bifunctional polyethylene glycol (COOH-PEG-SH) chain and subsequently attaching the formed IgG-PEG-SH onto AuNRs surface through an Au—S linkage. The PEG chain, served as a flexible spacer, increased the accessibility of IgG to target biomolecules and also formed a stable protective layer around AuNRs to minimize non-specific binding. After conjugating IgG onto AuNRs surface, the longitudinal LSPR wavelength of the AuNRs exhibited a red shift of 7.5 nm due to the increase in the refractive index of the medium surrounding AuNR (FIG. 2B). The AuNR-IgG conjugates were then adsorbed onto the 3-mercaptopropyl)trimethoxysilane-modified glass substrates. To probe the sensing capability of the plasmonic nanobiosensor, the substrates were exposed to different concentrations of anti-IgG solution leading to its specific binding to IgG, which was quantified by the red-shift in the LSPR wavelength of AuNRs.

A monotonic increase in the LSPR shift with an increasing concentration of the anti-IgG was observed. The limit of detection was found to be 240 μg/ml (FIG. 2C). Considering that the LSPR wavelength red shifted maximally by 15.5 nm at the highest concentration (24 μg/ml) of anti-IgG (FIG. 2D), this concentration was used to quantify the biorecognition capability of antibody (IgG) at elevated temperatures and extended incubation times.

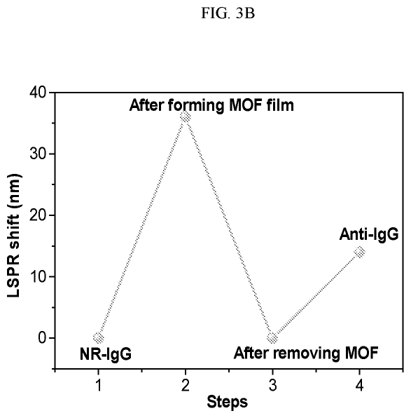

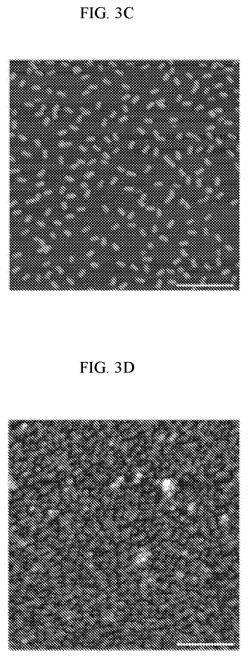

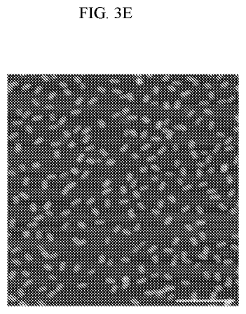

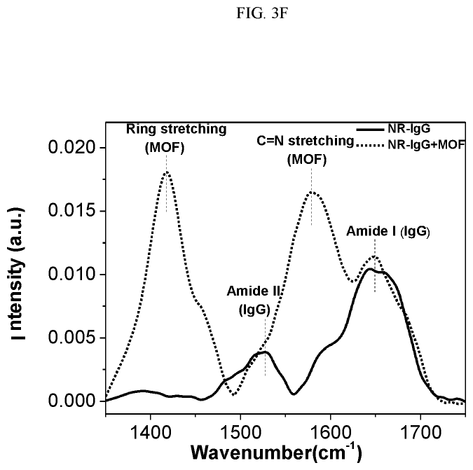

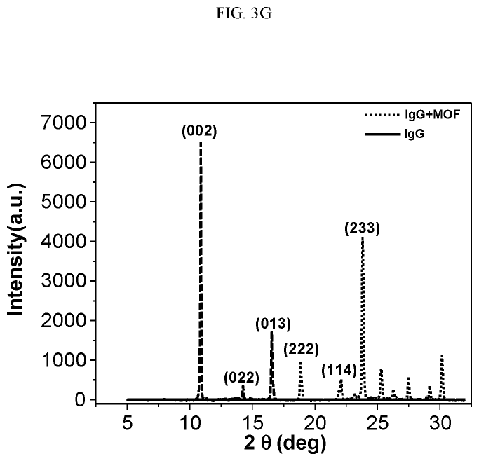

Apart from the detection of target analyte, LSPR shift of AuNRs was used to examine the formation and removal of the MOF film (FIGS. 3A and 3B). After immersing the biochips with immobilized AuNR-IgG into MOF precursor solution for 3 hours, the LSPR wavelength exhibited a ˜30 nm red shift (step 2 in FIG. 3B), indicating the formation of MOF film on top of the AuNR-IgG conjugates. After storing the MOF-coated plasmonic biochips at the desired temperature for the desired duration, the MOF protective coating was quickly removed by rinsing the biochip with water at pH 6. The complete removal of the MOF was evidenced by a ˜30 nm blue shift in the LSPR wavelength (step 3 in FIG. 3B). Removal of the MOF protective layer before exposure to the analyte solution exposes the active binding sites of the antibody and restores the recognition capability of the biosensor. Subsequently (step 4 in FIG. 3B), the restored substrate exhibited a red shift of 14 nm upon specific binding of anti-IgG (24 μg/mL) to IgG.

To quantitatively evaluate the antibody preservation efficiency at different elevated temperatures after several days of storage, the percentage of retained recognition capability (%) was used. The retained recognition capability was calculated as the percentage of the red shift upon specific binding of anti-IgG (24 μg/mL) to IgG on a restored biochip after several days of storage at elevated temperatures compared to the red shift obtained from the same batch of freshly-made substrate (which was considered as the reference sample tested instantly without MOF coating). As shown in FIGS. 3A and 3B, the red shift of 14 nm compared to the red shift of 16 nm obtained from the reference sample in the same batch corresponds to a retained recognition capability of 87.5%. Thus, 87.5% recognition capability of the antibody-based biosensor was found to be preserved after two days of storage at room temperature.

The MOF film formation and removal was further confirmed by atomic force microscope (AFM) imaging. Prior to incubating the substrate into MOF precursor solution, AFM imaging showed a uniform distribution of AuNR-IgG conjugates on the glass surface (FIG. 3C). After MOF film formation, the AuNR-IgG conjugates were completely covered by the MOF film as evidenced by the dense grainy morphology in the AFM image (FIG. 3D). After rinsing the substrate with water at pH 6, the AuNR-IgG conjugates were exposed without any MOF residue on the substrate (FIG. 3E).

To ascertain that ZIF-8 crystals were formed on the surface of AuNR-IgG conjugates, Fourier transform infrared spectroscopy (FTIR) and X-ray diffraction (XRD) were employed (FIGS. 3F and 3G). FTIR spectrum (black line in FIG. 3F) obtained before the formation of the MOF layer exhibited absorption peaks at 1640-1650 cm⁻¹ and 1520-1530 cm⁻¹, corresponding to amide I and amide II bands of IgG, respectively. After the MOF formation (red line in FIG. 3F), the FTIR spectrum exhibited new absorption bands corresponding to MOF apart from the amide I and amide II bands of the protein. The characteristic absorption peak at 1583 cm⁻¹ corresponds to the C═N stretching of imidazole and the peak at 1420 cm⁻¹ associated with the imidazole ring stretching. Interestingly, the amide I vibrational mode for the MOF-coated AuNR-IgG shifted to higher frequency (from 1643 to 1649 cm⁻¹), suggesting the protein-MOF interaction due to the coordination between the Zn cations and the carbonyl group of the proteins. Furthermore, x-ray diffraction (XRD) measurements confirmed the formation of ZIF-8 crystals on the top of AuNR-IgG surface (FIG. 3G). The XRD patterns and related peak positions were in agreement with typical structure of ZIF-826-27 except the absence of (011) plane, implying the preferential orientation of ZIF-8 formed on AuNR-IgG immobilized substrate.

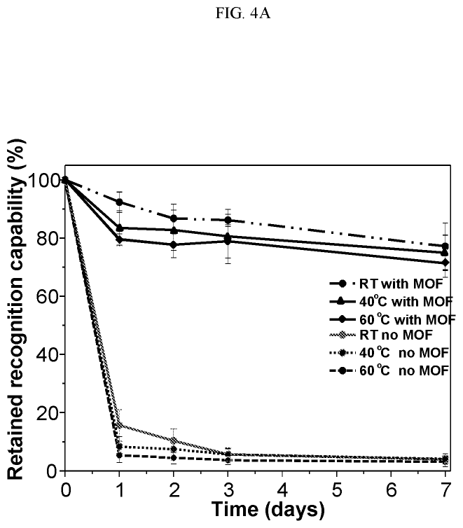

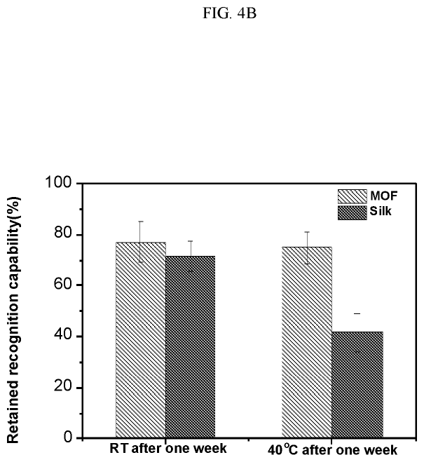

To determine the efficacy of MOF to preserve the biorecognition ability of IgG conjugated to AuNR upon exposure to harsh conditions (such as high temperatures) that would normally lead to denaturation and loss of recognition capabilities, the plasmonic biochip with and without MOF coating were stored at room temperature, 40° C. and 60° C. for one week. Different substrates were sampled at selected time intervals (1 day, 2 days, 3 days and 7 days) to monitor the changes in the biorecognition capability of the antibodies (FIG. 4A).

Samples with MOF coatings showed only a ˜20% loss in biorecognition capability after storage at room temperature (25° C.) for one week compared to the nearly complete loss in biorecognition capability for substrates without MOF protective layer. Even at higher temperatures, 40° C. and 60° C., MOF-coated biochips retained over 70% of biorecognition ability after one week. In contrast, substrates without a MOF protective layer lost over 90% of biorecognition capability within the first day 40° C. and 60° C. Compared AuNR using silk fibroin as a protective layer, MOF coating was superior in terms of biopreservation efficacy and ease of formation and removal. While the silk-coated plasmonic biochips retained a biorecognition capability of ˜40% after one week at 40° C., the MOF-coated biochips retained over 70% of biorecognition ability under identical storage conditions (FIG. 4B).

The applicability of this approach to clinically-relevant biodiagnostic devices was evaluated. Neutrophil gelatinase-associated lipocalin (NGAL), a urinary biomarker for acute kidney injury, was selected as the target analyte. Considering that urinary NGAL levels are increased by several log-orders (100 to 1,000-fold) of magnitude during acute kidney injury, rapid measurement of urine NGAL levels in resource-limited settings is of great clinical importance.

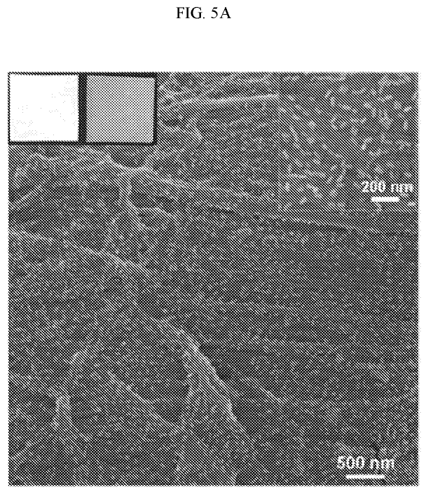

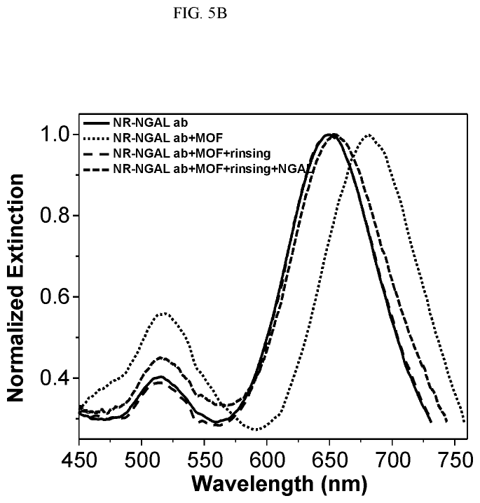

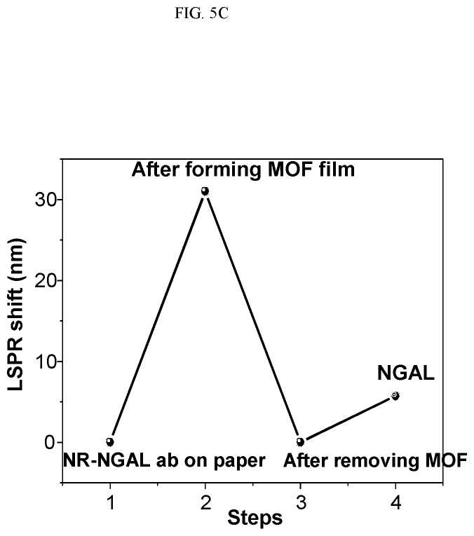

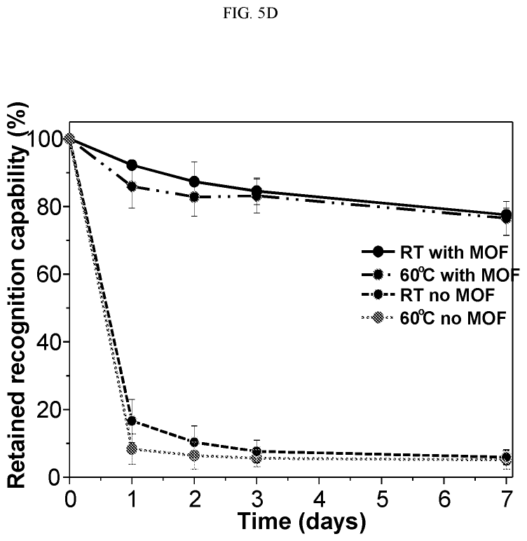

The fabrication a bioplasmonic paper device (BPD) for NGAL detection was achieved by the immersion of a 1×1 cm strip of filter paper in the solution of NGAL antibody-conjugated AuNR. The SEM images of the paper revealed a uniform distribution of the AuNR-NGAL antibody conjugates with no signs of aggregation or patchiness (FIG. 5A). Similar to AuNR-IgG on glass substrates, the coating of MOF on the paper substrate also induced ˜30 nm red shift (FIG. 5B). After rinsing with water at pH 6, a blue shift of ˜30 nm indicated the complete removal of MOF on the paper (FIGS. 5B and 5C). Time-lapse experiments, similar to the one described above, was performed to monitor the recognition capability of the NGAL antibodies on the paper-based plasmonic biosensor stored at room temperature and 60° C. As demonstrated above, the antibody-based BPDs with a MOF protective coating retained nearly 80% of recognition capability after one week of storage at both room temperature and 60° C., while bare BPDs without a MOF protective layer lost activity quickly at both temperatures. Apart from the generality of the MOF protection approach, these results demonstrate the feasibility of dramatically enhancing the thermal stability and preserving the recognition capability of a clinically relevant biosensor device, enabling their use in POC and resource-limited settings.

These results demonstrate that MOF (ZIF-8) can be used as a protective material to preserve the recognition capability of antibodies on biosensor surfaces stored at ambient and elevated temperatures. With the protection of MOF, both rabbit IgG and anti-NGAL antibodies on plasmonic biosensors retained over 70% of recognition capability (compared to complete loss in unprotected samples) after one week of storage at room temperature, 40° C. and 60° C. Such antibody-based biosensors with enhanced thermal stability eliminate the need for cold chain system during transportation and storage in an energy-efficient and environmentally-friendly fashion. Furthermore, the biofunctionality of the MOF-coated biochip can be restored by a simple water rinsing step, making it highly convenient for use in POC and resource-limited settings such as an ambulance, intensive care unit, emergency department, battlefield and developing world. The results also demonstrate generality and applicability of this approach to a clinically-relevant bioplasmonic paper device, which offers several advantages over rigid substrates. The high preservation efficiency and ease of implementation (formation and removal) makes of MOFs vastly superior to our previous silk-based preservation approach. Overall, we expect this facile and low-cost biopreservation method to greatly advance the application of various antibody-based biosensor platforms in POC and resource-limited settings.

Owing to the high sensitivity, cost-efficiency and great potential of use as point-of-care (POC) diagnostics, the plasmonic nanobiosensor based on refractive index sensitivity of LSPR is used as the platform to monitor fabrication stages including antibody conjugation, MOF formation and removal, as well as for bioanalyte detection. Proof of concept is established by using IgG/anti-IgG as a model system, showing that MOF layer remarkably improves the stability of model antibody at room temperature, 40° C. and 60° C. The biopreservation efficiency of MOFs is found to be higher than the previously reported silk-based approach. This approach can be applied to increase the shelf-life and thermal stability of a bioplasmonic paper device designed for the detection of biomarkers in resource-limited settings. Overall, by eliminating the cold chain requirement in transportation, storage and handling through an environmentally-friendly and energy-efficient method, this novel approach paves way for antibody-based biosensors in POC and resource-limited settings such as ambulance, developing countries, battlefield and patient's home.

### Example 2

In this Example, metal-organic frameworks were analyzed for signal enhancement in artificial antibody based plasmonic biosensors.

AuNRs were synthesized using a seed-mediated approach as described above (illustrated in FIG. 6). Protein immobilization was performed by exposing the functionalized substrates to human serum albumin template (HSA) prepared in PBS solution (pH 8.3). The exposure was performed under gentle shaking for 30 min followed by 2 hour incubation at 4° C. Following the immobilization of the template, organo-siloxane monomers trimethoxypropylsilane (TMPS) and (3-aminopropyl)trimethoxysilane (APTMS) were co-polymerized onto the modified AuNR surface. The subsequent condensation of the transient silanol groups yields an aminopropyl-functional amorphous polymer and entrapment of the protein templates. After rinsing, the protein-coated AuNR were immersed in 3 mL phosphate buffer (pH 7.5) to which 5 μL TMPS and 5 μL APTMS were freshly added. The samples were then gently rinsed with PBS solution (pH 7.5) and stored in the same buffer overnight at 4° C. The protein template inside the siloxane co-polymer was released by breaking the imine bonds of the cross linker using a mixture of 2% SDS and 2 mM oxalic acid for 1 hour. The AuNR plasmonic biosensors with artificial antibodies formed by the molecular imprint approach were then incubated with a sample to capture protein. Following protein capture, mineralization of MOF around the captured protein was performed to enhance the LSPR signal.

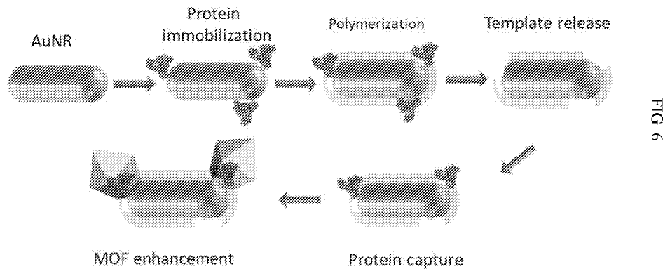

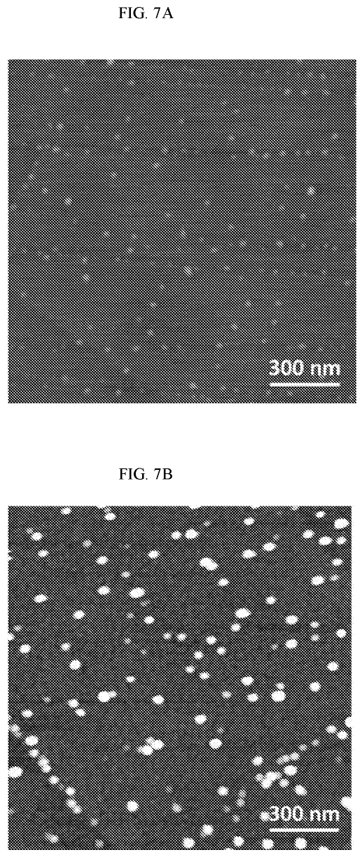

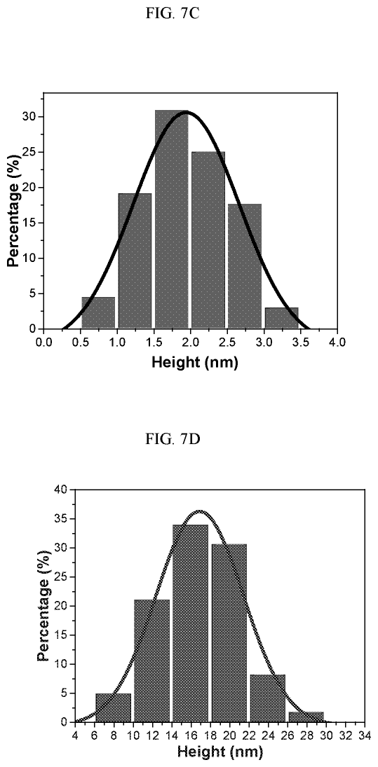

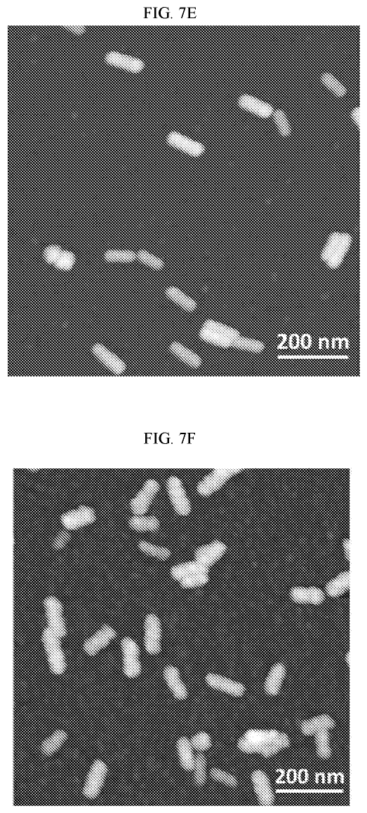

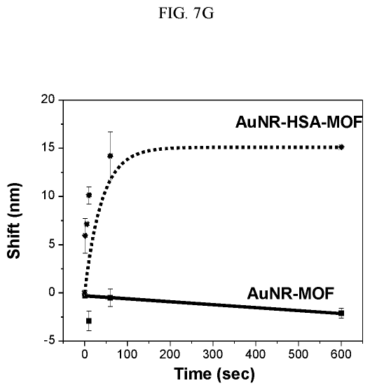

AFM height images of HSA absorbed on HOPG substrate (height scale: 15 nm) (FIG. 7A) and HSA after ZIF-8 growth (height scale: 30 nm) (FIG. 7B) (corresponding height histograms shown in FIG. 7C and FIG. 7D). AFM height images showing AuNR absorbed with HSA (FIG. 7E) and after ZIF-8 growth (FIG. 7F) showed surfaces with significantly higher roughness. FIG. 7G depicts the LSPR shift induced by the formation of MOF vs. growth time on HSA modified AuNR and bare AuNR.

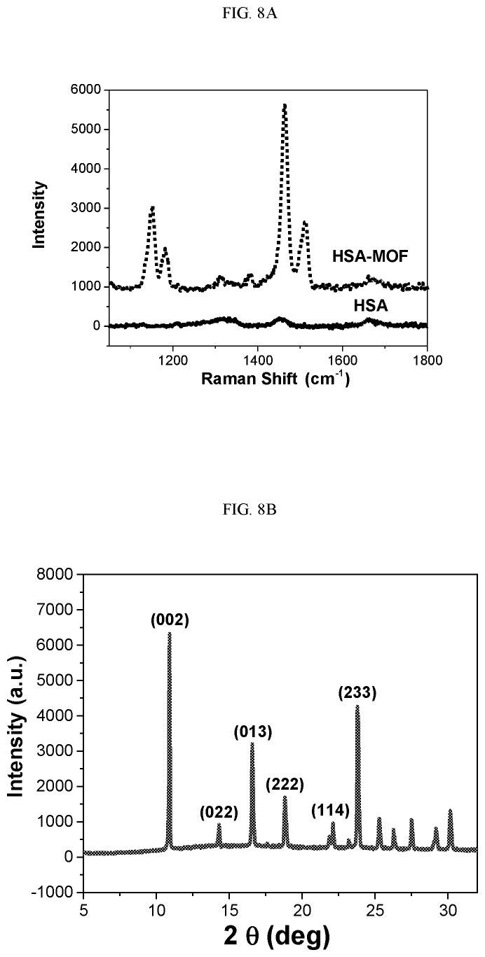

FIG. 8A depicts Raman spectra of HSA before (HSA) and after AuNR plasmonic biosensors were exposed to ZIF-8 precursor. FIG. 8B depicts x-ray diffraction (XRD) of HSA before (HSA) and after AuNR plasmonic biosensors were exposed to ZIF-8 precursor.

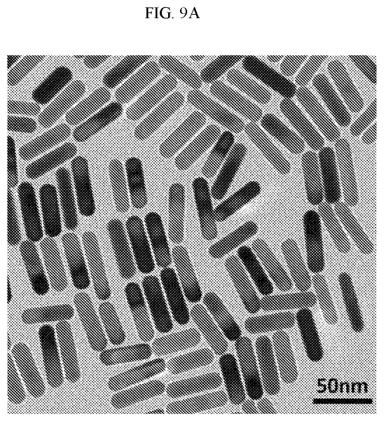

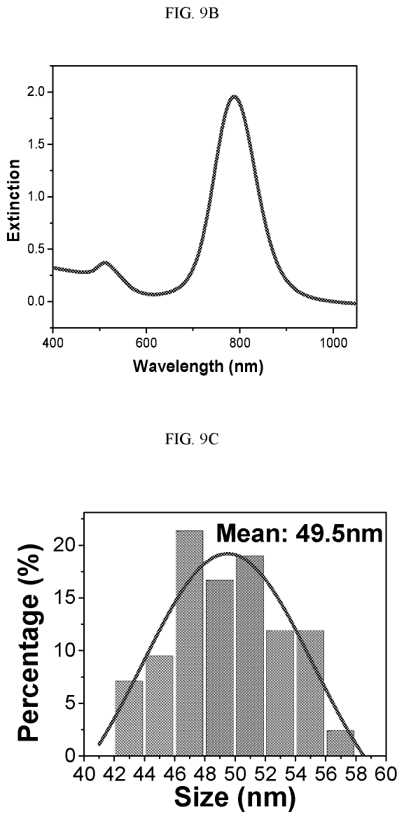

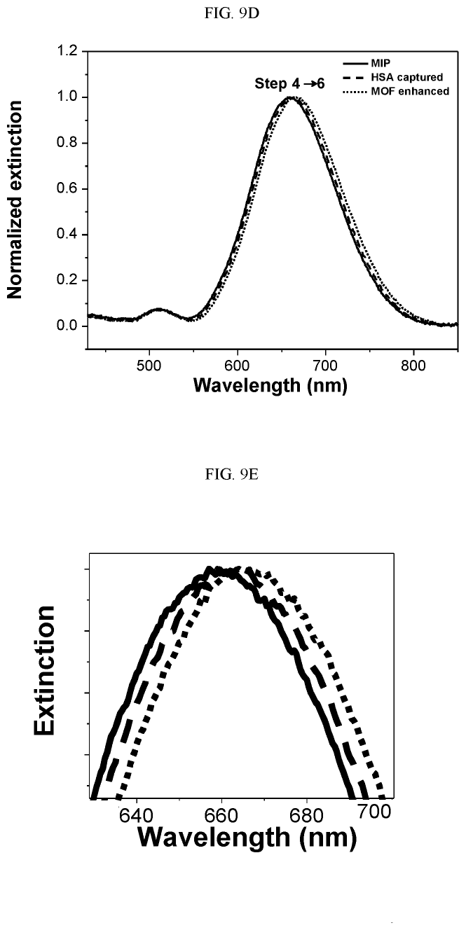

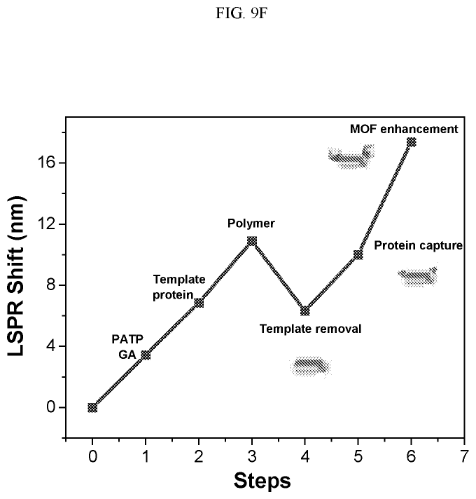

FIG. 9A is a TEM image of AuNR used as nanotransducers for artificial antibody-based biosensors. FIG. 9B depicts Vis-NIR extinction spectra of an aqueous suspension of AuNR. FIG. 9C depicts the size histogram of AuNR obtained from TEM image, revealing the average length of AuNR˜49.5 nm. FIG. 9D is a graph depicting the extinction spectra exhibiting a continuous red shift of LSPR wavelength of MIP-AuNR biosensor after protein capture, and ZIF-8 mineralization. FIG. 9E is an enlargement of the curve in FIG. 9D between 620 nm and 700 nm. FIG. 9F depicts the LSPR shift of AuNR after each step along the fabrication of MIP, target protein capture and MOF enhancement.

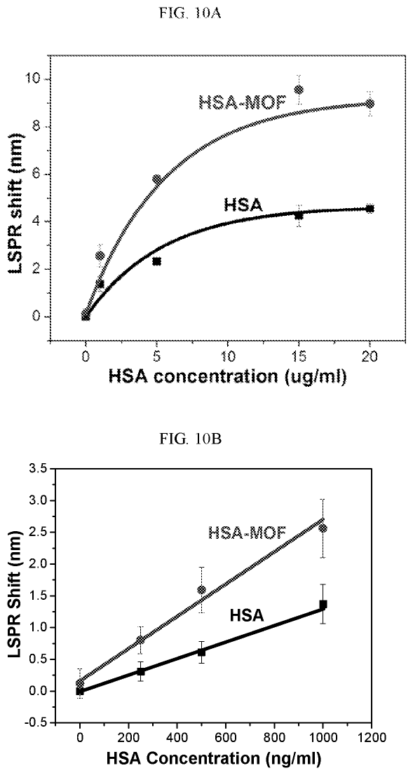

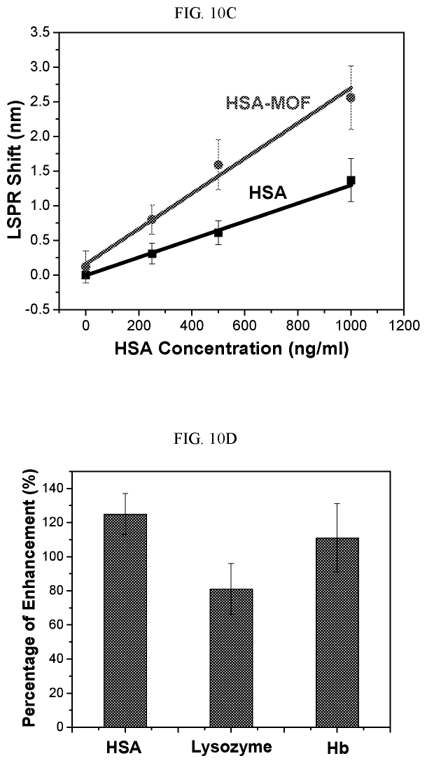

FIG. 10A is a graph depicting the LSPR shift vs. concentration of the target protein biomarker (HSA) before and after MOF amplification. FIG. 10B is a graph depicting the concentration dependent LSPR shift for normal AuNR-MIP and for MOF amplified AuNR-MIP at low target protein concentration. FIG. 10C is a graph depicting the LSPR shift induced by the formation of MOF vs. MOF growth on molecularly imprinted AuNR before and after target protein capture. FIG. 10CD is a graph depicting MOF amplification of MIP-based plasmonic for three different proteins biosensors showing the generality of the signal amplification strategy.

### Example 3

In this Example, paper-based plasmonic biosensors were prepared for detecting Zika virus.

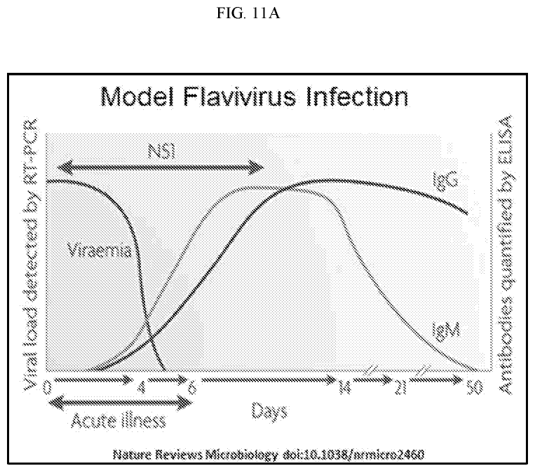

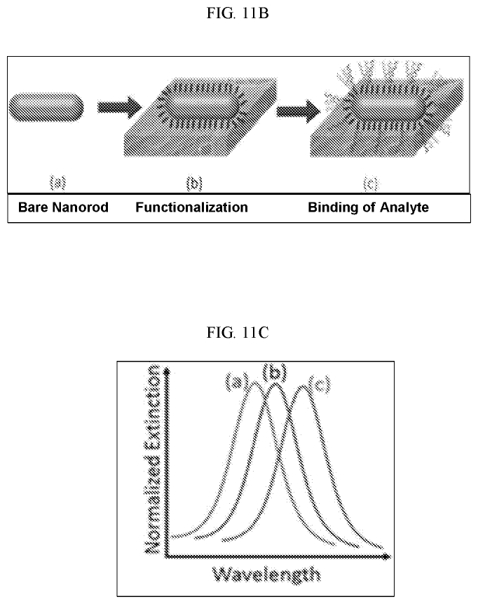

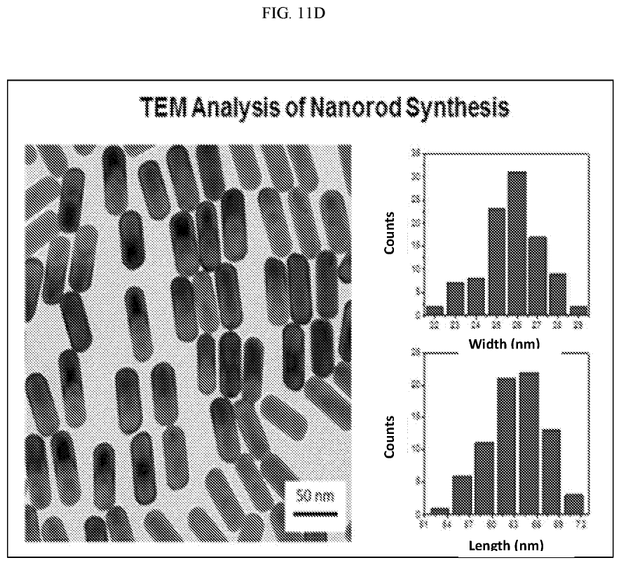

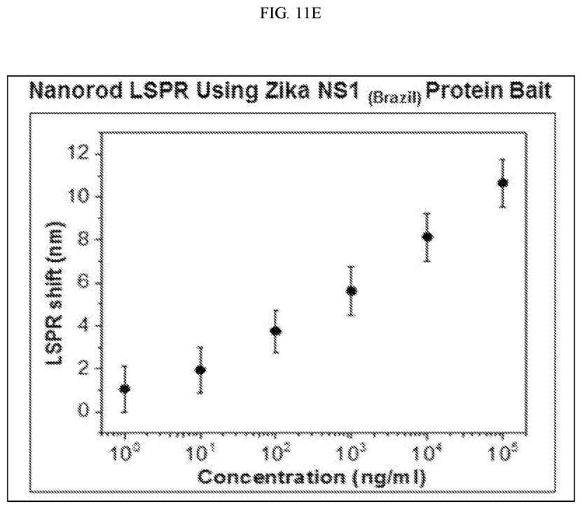

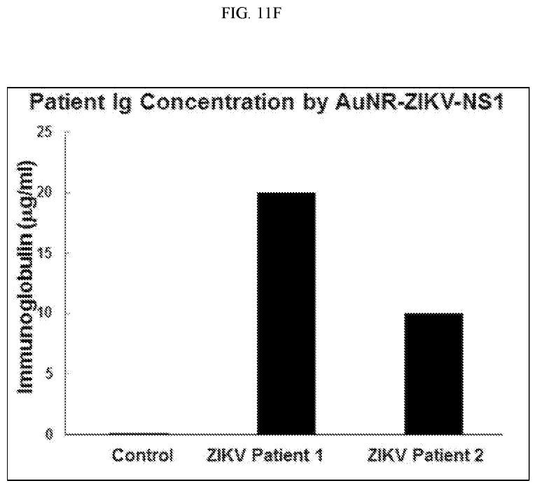

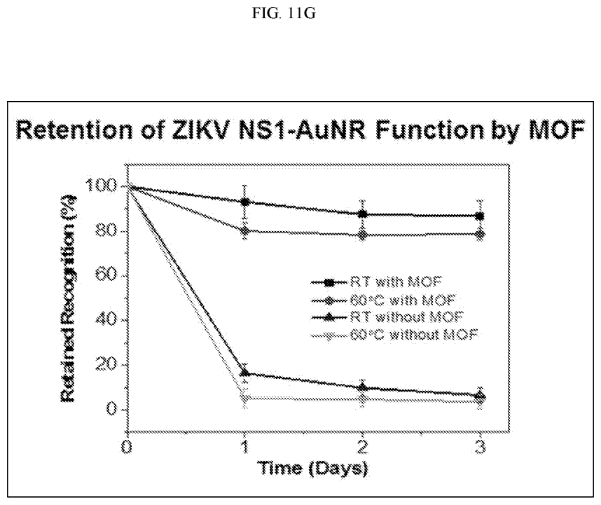

Viral NS1 protein appears transiently in the plasma/sera within days of infection. This causes an IgM and IgG immune response in the patient which appears in days and persists for several weeks or months. Detecting the NS1-directed IgM and IgG is the basis for the assay as shown in FIG. 11A.

AuNR were synthesized as described above. As illustrated in FIG. 11B, Zika NS-1 protein was conjugated to bare AuNRs (step (a)) as described above for IgG molecules. After conjugating Zika NS-1 protein onto AuNRs surface (step (b)), the AuNRs were deposited on Whatman 1 paper to create substrates. Substrates were then incubated with a monoclonal IgG antibody specific for Zika NS-1 protein (sept (c)) in buffer or 10% human sera to generate standard curves of immune IgG bound to the substrate. Antibody binding was measured in steps (a)-(c) by a red-shift of the LSPR of the AuNR (FIG. 11C). FIG. 11D depicts the homogeneity of the nanorod preparation prior to functionalization with recombinant Zika NS1 protein “bait”. Documented Zika-positive patient sera were obtained from Antibody Systems Inc. (Hurst, Tex.). Sera from 5 control patients and 2 ZIKV-infected patients were analyzed using AuNR-ZIKV-NS1 biochips. As little as 1 ng/ml of NS-1 IgG was detected up to concentrations of 100 μg/ml (FIG. 11E). FIG. 11F depicts the concentration of IgM/IgG in control Zika-naive individuals and two Zika-infected patients. This shows the clinical utility of the ZikaNS1-AuNR to detect patients infected by Zika virus.

Functionalized rods with/without MOF were exposed to nominal room temperature (20° C.) or 60° C. for one or three days. FIG. 11G represents the percent retained binding activity of IgG1 based upon the 10 nm shift in 10 μg/ml monoclonal IgG in LSPR.

The plasmonic devices and a metal-organic framework protected plasmonic sensors of the present disclosure provide an effective platform for preserving biomolecules under extreme storage conditions. Apart from facile formation of the MOF protective coatings, a simple water rinsing step can restore the biofunctionality of the MOF-coated plasmonic devices and MOF-protected plasmonic sensors, making it highly convenient for use in point-of-care and resource-limited settings. This energy-efficient and environmentally-friendly approach can reduce or eliminate the need for a temperature-controlled supply chain (the “cold chain”) and, temperature-controlled packing and transport of diagnostic reagents and materials, thereby extending the capability of biomolecule-based biosensors to various resource-limited circumstances.

The metal-organic framework enhanced plasmonic sensors of the present disclosure also allow for enhanced signal amplification of following target analyte capture. Signal amplification by metal-organic framework enhanced plasmonic sensors of the present disclosure is particularly advantageous where low target analyte concentrations exist.

In view of the above, it will be seen that the several advantages of the disclosure are achieved and other advantageous results attained. As various changes could be made in the above devices and methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A metal-organic framework protected plasmonic sensor comprising a nanostructure core, a biomolecule, and a metal-organic framework.

2. The metal-organic framework protected plasmonic sensor of claim 1, wherein the nanostructure core comprises a metal nanoparticle.

3. The metal-organic framework protected plasmonic sensor of claim 2, wherein the metal nanostructure core comprises at least one of a nanosphere, a nanorod, a nanotube, a nanocube, a nanobipyramid, a nanocages, a nanostar, a nano-octahedron, a nanorattle, a nanoshell, a nanomatryoshka and combinations thereof.

4. The metal-organic framework protected plasmonic sensor of claim 2, wherein the metal nanostructure core comprises a gold nanostructure core.

5. (canceled)

6. The metal-organic framework protected plasmonic sensor of claim 1, wherein the metal-organic framework comprises a ZIF-8 metal-organic framework.

7. The metal-organic framework protected plasmonic sensor of claim 1, wherein the biomolecule comprises at least one of a nucleic acid, a full-length protein, a polypeptide, a peptide, a lipid, a glycolipid, an aptamer, and combinations thereof.

8-25. (canceled)

26. A plasmonic device comprising a substrate and a metal-organic framework protected plasmonic sensor, wherein the metal-organic framework protected plasmonic sensor comprises a nanostructure core, a biomolecule, and a metal-organic framework.

27. The plasmonic device of claim 26, wherein the nanostructure core comprises a metal nanoparticle.

28. The plasmonic device of claim 27, wherein the metal nanostructure core comprises at least one of a nanosphere, a nanorod, a nanotube, a nanocube, a nanobipyramid, ananocages, a nanostar, a nano-octahedron, a nanorattle, a nanoshell, a nanomatryoshka and combinations thereof.

29. The plasmonic device of claim 27, wherein the metal nanostructure core comprises a gold nanostructure core.

30. The plasmonic device of claim 26, wherein the metal-organic framework comprises a ZIF-8 metal-organic framework.

31. The plasmonic device of claim 26, wherein the biomolecule comprises at least one of a nucleic acid, a full-length protein, a polypeptide, a peptide, a lipid, a glycolipid, an aptamer, and combinations thereof.

32. A method for enhancing a signal produced in a label-free detection method, the method comprising:
contacting a sample with a plasmonic sensor, wherein the plasmonic sensor comprises a nanostructure core and functional monomers polymerized to the nanostructure core, wherein the polymerized functional monomers comprise at least one recognition cavity that is substantially complementary to a target analyte; wherein a target analyte in the sample forms a complex with the plasmonic sensor;
incubating the complex in a metal-organic framework precursor solution and;
detecting the complex.

33. The method of claim 32, wherein the complex is detected using a method selected from the group consisting of local surface plasmon resonance and surface enhanced Raman scattering.

34. The method of claim 32, wherein the nanostructure core is selected from the group consisting of a gold nanostructure core, a silver nanostructure core, a copper nanostructure core, and combinations thereof.

35. The method of claim 32, wherein the sample comprises a liquid biological sample.

36. The method of claim 35, wherein the liquid biological sample is selected from the group consisting of whole blood, plasma, serum, urine, saliva, cerebrospinal fluid, interstitial fluid, and sweat.

37. The method of claim 32, wherein the target analyte is selected from the group consisting of a cell, a metabolite, a protein, a peptide, a nucleic acid, and combinations thereof.

38. The method of claim 32, further comprising adsorbing the plasmonic sensor to a substrate prior to contacting the sample with the plasmonic sensor.

39. The method of claim 38, wherein the substrate is selected from the group consisting of a glass substrate, a paper substrate, a plastic substrate and a fibrous mat.
